(19) United States
(12) Patent Application Publication
(10) Pub. No.: US 2003/0087422 A1
(43) Pub. Date: May 8, 2003

(54) Biogenic FPA: enzymatic production of formylphosphonic acid by the oxidation of hydroxymethylphosphonic acid

(75) Inventors: Michael A. Heitkamp, Aiken, SC (US); B. Mason Hughes, St. Louis, MO (US); Shekhar Viswanath, St.Louis, MO (US)

Correspondence Address: JENKENS & GILCHRIST, A PROFESSIONAL CORPORATION, 1100 LOUISIANA, SUITE 1800, HOUSTON, TX, 77002-5214, US

(73) Assignee: Monsanto Technology LLC.

(21) Appl. No.: 10/196,662

(22) Filed: Jul. 15, 2002

Related U.S. Application Data
(60) Provisional application No. 60/306,553, filed on Jul. 19, 2001.

(51) Int. Cl. C12S 1/00; C12N 1/21

(52) U.S. Cl. 435/262.5; 435/252.3

(57) ABSTRACT

Pure bacterial cultures for the production of formylphosphonic acid (FPA) by the oxidation of hydroxymethylphosphonic acid (HMPA) are provided. The cultures can be used to bioremediate HMPA contaminated wastewater or soil, or can be used to produce biogenic FPA for subsequent use. The production of glyphosate from biogenic FPA is described.

### PRIOR RELATED APPLICATIONS

This application claims priority to prior U.S. Provisional Patent Application Serial No. 60/306,553, filed Jul. 19, 2001, which is incorporated by reference herein in its entirety.

### FEDERALLY SPONSORED RESEARCH STATEMENT

Not applicable.

### REFERENCE TO MICROFICHE APPENDIX

Not applicable.

### FIELD OF THE INVENTION

The present invention provides the first known report of formylphosphonic acid (FPA) produced enzymatically from hydroxymethylphosphonic acid (HMPA). Studies utilized a mixed microbial culture in an aerobic stirred bioreactor acclimated to HMPA for seven months. Microbial plating studies isolated several pure bacterial cultures showing HMPA degradation and FPA production. Hydrogenation of a HMPA-degrading mixed bacteria culture to which glycine was added resulted in production of 81 mg/L of N-phosphonomethylglycine (glyphosate) from the FPA.

### BACKGROUND OF THE INVENTION

Glyphosate is a broad-spectrum herbicide widely used to kill unwanted plants both in agriculture and in nonagricultural landscapes. Estimated use in the U.S. is between 19 and 26 million pounds per year and glyphosate is the active ingredient of the herbicide ROUNDUP™ (MONSANTO™).

Glyphosate inhibits the shikimic acid pathway in plants and some microorganisms. The shikimic acid pathway provides a precursor for the synthesis of aromatic amino acids and other secondary metabolites. Specifically, glyphosate inhibits the conversion of phosphoenolpyruvate and 3-phosphoshikimic acid to 5-enolpyruvyl-3-phosphoshikimic acid by inhibiting the enzyme 5-enolpyruvyl-3-phosphoshikimate synthase. As a consequence of the inhibition of aromatic amino acid biosynthesis, protein synthesis is disrupted, resulting in the plant's death.

Glyphosate is produced by chemical synthesis and is not believed to be a natural product. It can be degraded, however, by enzymatic means in several microorganisms. Chemically, glyphosate is N-phosphonomethyl-glycine:

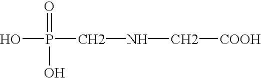

Glyphosate can be chemically synthesized in a number of ways (see e.g., U.S. Pat. Nos. 3,927,080, 3,969,398, 4,065,491, 4,486,359, 4,851,159, 4,983,764, 5,874,612, 6,005,140), but few, if any, methods exist for the enzymatic synthesis of glyphosate or its precursors. U.S. Pat. No. 05,180,846, however, describes a process of making glyphosate by hydrogenating a mixture containing glyoxylic acid and aminomethylphosphonic acid, the mixture having been enzymatically prepared in situ by the reaction of glycolic acid and oxygen in an aqueous solution containing aminomethylphosphonic acid and the enzymes glycolate oxidase and catalase. This is the only known example of the enzymatic synthesis of a immediate precursor for glyphosate.

One glyphosate synthesis involves a precursor primary or secondary amine, such as glycine, that is condensed with a carbonyl compound, such as formylphosphonic acid (FPA) or its acetals. The condensation product is then reduced to produce the desired product as described in U.S. Pat. No. 4,568,432. This reaction is illustrated in FIG. 1.

Although there are chemical means of preparing FPA (e.g., U.S. Pat. No. 6,054,608), one desirable method would be the enzymatic production of FPA from a precursor molecule, such as hydroxymethylphosphonic acid (HMPA). This pathway would be of particular interest because it utilizes HMPA as a low-cost intermediate produced almost quantitatively from formaldehyde and phosphorous acid. The initial chemical reaction of formaldehyde and phosphorous acid to produce HMPA and the final hydrogenation of the condensation product of FPA and glycine to produce glyphosate are both well-established and efficient reactions. Thus, the critical need for this promising new approach is the discovery of a cost-effective intermediate step to oxidize HMPA to FPA. This promising new route would be efficient, cost-effective and would produce less waste than current glyphosate manufacturing techniques. However, no biochemical pathways have been described to date that allow the production of FPA and thus the invention fulfills this long felt need in the art.

### SUMMARY OF THE INVENTION

Abbreviations and Definitions

Biogenic FPA—FPA that is produced by enzymatic activity

Derivatives—includes all salts of a compound

FPA—formylphosphonic acid and salts thereof

FPA derivatives—compounds according to Formula II or Formula V in FIG. (includes acetal & hemiacetal forms which are in equilibrium)

HMPA—hydroxymethylphosphonic acid and salts thereof

HMPA derivatives—compounds according to Formula I in FIG. 5

HMPA Oxidase—the enzyme that converts HMPA to FPA

Glyphosate—N-phosphonomethylglycine and salts thereof

Glyphosate derivative—compounds as disclosed in U.S. Pat. Nos. 4,405,531, 3,835,000 and shown by Formula IV in FIG. 5. See also amino phosphonate derivative.

Nitrogen-containing compounds—the compounds shown by Formula III of FIG. 5, includes glycine

Generally speaking, the invention relates to novel microbes that have HMPA degrading activity. The enzyme or enzymes responsible for this activity have not yet been characterized, but is suspected to be a single enzyme that is designated herein “HMPA oxidase.” HMPA oxidase activity may also require the activity of multiple proteins.

In addition, a method of isolating additional pure cultures of HMPA degrading bacteria are exemplified. The method requires seeding a test reactor with mixed microbial samples, and placing the microbes under selective pressure by using HMPA as the sole carbon source in the reactor. When HMPA degrading activity is detected, individual clonal isolates of such bacteria are prepared by standard techniques.

The discovery of HMPA oxidase activity enables a number of previously unknown enzymatic methods. For example, a method of bioremediating wastewater or soil samples contaminated with HMPA is now possible. This method involves exposing contaminated media to one or more HMPA degrading microbe under conditions that allow the degradation of HMPA. Further, the HMPA degrading microbe may be selected from the group consisting of ATCC ______, ATCC ______, and ATCC ______ or may be a mixed HMPA degrading microbial sample at ATCC PTA-3469 (named HMPA-M1 and deposited Jun. 19, 2001). In this application, mixed cultures may be preferred as being more robust and genetically diverse.

Another invention enabled by the discovery of HMPA oxidase is an enzymatic method of producing FPA. Enzymatically produced FPA is designated as “biogenic FPA” herein. The method requires incubating HMPA with one or more HMPA degrading microbe under conditions that allow the degradation of HMPA to FPA and collecting the FPA. The collection may be by trapping the FPA or siphoning it continuously out of the system. However, where a mutant microbe that accumulates FPA is used, the FPA may be harvested in the usual manner of lysing the cells and purifying the FPA.

A method of producing glyphosate by condensing biogenic FPA and glycine and subsequent conversion to glyphosate by reduction is also described. It is also possible to perform this reaction in situ by trapping the FPA in the culture media with glycine and subsequent reduction. Alternatively, the FPA may be reacted with ammonia to produce aminomethylphosphonic acid (AMPA), which is then converted to glyphosate by reduction, carboxymethylation, oxidation, or combinations thereof, as is already known in the art.

HMPA and its derivatives are represented by Formula I in FIG. 5. R1 and R2 individually are hydrogen, hydrocarbyl, substituted hydrocarbyl, a salt-forming cation, or a heterocycle. HMPA may be produced by any method. For example, HMPA can be prepared by reacting formaldehyde with phosphorus acid as disclosed in U.S. Pat. No. 5,266,722 and GB 1076244.

FPA and its derivatives are represented by Formula II of FIG. 5. R1 and R2 are the same as in Formula I. The FPA compound of Formula II or its hydrate, hemiacetal, or acetal (see e.g., Formula V of FIG. 5) produced in embodiments of the invention have many useful applications. It may be used as an end product or an intermediate for making glyphosate or other valuable fine chemicals.

Any method that converts FPA to a glyphosate derivative in one or more steps may be used in embodiments of the invention. For example, a glyphosate derivative may be produced by first reacting FPA with a nitrogen-containing compound to obtain a condensation product. The condensation product can then be converted to a glyphosate derivative by one or more reactions, such as hydrogenation, carboxymethylation and/or oxidation. A detailed description of the production of glyphosate derivatives from HMPA derivatives is found in copending application Ser. No. 09/728,577, incorporated by reference herein.

One class of suitable nitrogen-containing compounds is represented by Formula III of FIG. 5. The n can be 0, 1, 2, 3, 4, 5, 6, or other positive integers. R3 is —H, —OH, —NH₂, —CONH₂, —CO₂H (provided that when R is —COOH, n is not zero). Preferably, n is 0, 1, or 2. When n is 0 and R3 is hydrogen or —CO—NH₂, the compound is ammonia or urea respectively. When n is 2 and R3 is —OH, the compound is ethanolamine. When n is 1 and R3 is COOH the compound is glycine. In some embodiments, secondary amines may be used in embodiments of the invention in a manner as described in U.S. Pat. NO. 4,568,432.

The reaction between the formylphosphonic acid compound of Formula II and a primary amine or a salt thereof produces a condensation product. Upon hydrogenation, the condensation product is converted to a glyphosate compound. This reaction is shown in FIG. 1 and described in U.S. Pat. No. 4,568,432. The glyphosate product may be separated from the reaction mixture according to known methods.

Ammonia, its aqueous solution, or salts thereof (such as ammonium chloride or ammonium sulphate) may be used to react with FPA to form aminomethylphosphonic acid (AMPA) or salts thereof. AMPA or salts thereof may be carboxymethylated, i.e., reacted in any manner and in any number of steps to introduce a carboxymethyl moiety to form glyphosate or salts thereof.

The carboxymethylation reaction may be conducted in any manner so long as the AMPA is converted to glyphosate in one or more steps. For example, the carboxymethylation processes described in U.S. Pat. No. 4,422,982 and ES5044479 may be used. Any carboxymethylating agent may be used, including but not limited to, monohaloacetic acid or a salt thereof, monohaloacetamide, etc. The halogen is fluorine, chlorine, bromine, or iodine.

Other processes for converting AMPA to glyphosate may also be used. For example, U.S. Pat. Nos. 4,369,142 and 4,486,358 disclose a method for preparation of glyphosate by reacting AMPA with glyoxal in an aqueous medium and in the presence of sulfur dioxide gas. U.S. Pat. No. 5,453,537 discloses a method for making glyphosate by reacting AMPA with a glyconitrile in the presence of an alkali metal hydroxide to afford a product and then hydrolyzing the product by adding an alkali metal hydroxide in an amount sufficient to neutralize the resulting carboxylic acid. U.S. Pat. Nos. 5,578,190 and 5,874,612 disclose a method for making glyphosate by condensing AMPA with glyoxylic acid or a related aldehyde compound and reducing the resulting condensation product. U.S. Pat. Nos. 5,679,843 and 5,948,937 disclose a method for making glyphosate by reacting AMPA, an alkali metal cyanide (or hydrogen cyanide), and formaldehyde to form N-phosphonomethylglycinonitrile and hydrolyzing the N-phosphonomethylglycinonitrile. All of these processes are carboxymethylation methods with the end result of the addition of the carboxymethyl moiety.

Another synthetic route from FPA to glyphosate is to react FPA with ethanolamine or salts thereof to form a condensation product. The condensation product may be hydrogenated to hydroxyethylaminomethylphosphonic acid (HEAMPA) or salts thereof, which may be oxidized to glyphosate or salts thereof. Other suitable alkanolamines for producing useful compounds include, but are not limited to, 3-amino-1-propanol, 4-amino-1-butanol, 5-amino-1-pentanol, 6-amino-1-hexanol, etc.

The oxidation of HEAMPA to form glyphosate may be carried out in any manner. For example, HEAMPA may be oxidized by the oxidation processes described the prior art, such as the methods as described in U.S. Pat. Nos. 4,810,426, 5,292,936, and 5,602,276 or copending application Ser. No. 09/728,577 or ______ (electro-oxidation).

For commercial applications, glyphosate is generally employed in the form of a salt in which the cation is, for example, an alkali metal, an alkaline earth metal, ammonium, or an organic ammonium in herbicide formulations. The following U.S. patents disclose methods of making and using herbicide formulations for various applications: U.S. Pat. Nos. 3,799,580; 3,853,530; 3,977,860; 3,988,142; 4,140,513; 4,315,765; 4,405,531; 4,481,026; 4,507,250; 4,840,659; 5,693,593; 5,912,209; 5,935,905; 5,985,794; 5,994,269; 5,998,332; and 6,083,878. The glyphosate produced in accordance with embodiments of the invention can be utilized in a manner disclosed in the above patents.

### BRIEF DESCRIPTION OF THE DRAWINGS

FIG. 1. Glyphosate Synthesis from HMPA.

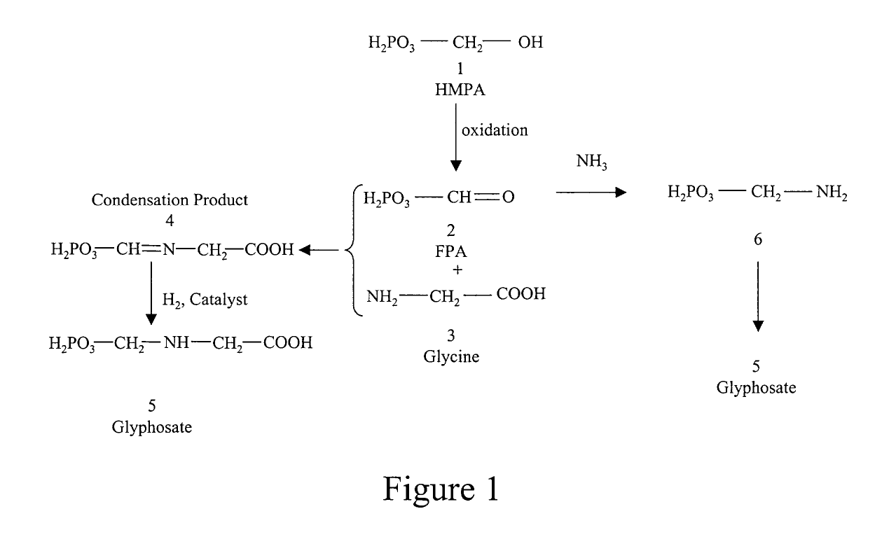

FIG. 2. HMPA Degradation for Microbial Isolates 2B, 3B, 5B, 6B, and 7B(2).

FIG. 3. Biodegradation of HMPA by Concentrated Mixed Microbial Cultures Collected from an Aerobic Stirred Reactor.

FIG. 4. FPA Degradation by Microbial Isolates 2B, 3B, 5B, 6B; and 7B(2).

FIG. 5. Formulae of the Invention.

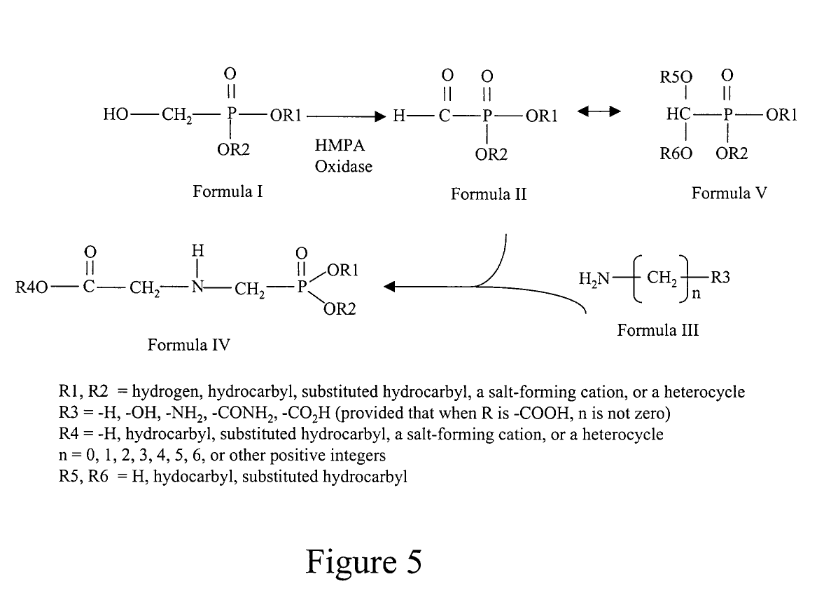

### DESCRIPTION OF EMBODIMENTS OF THE INVENTION

The development and utilization of microbial processes for biological treatment of glyphosate process wastewater has been well-established and important for achieving environmental compliance at glyphosate manufacturing facilities. Early work produced novel microbial degradation activities for glyphosate and glyphosate intermediate. This early work increased understanding of the fundamental microbial processes of glyphosate degradation and resulted in the development of reliable and efficient full-scale biological treatment systems for glyphosate wastes. It is noteworthy that this early work also led to the discovery and isolation of novel glyphosate-degrading bacteria expressing high levels of glyphosate oxidase. This discovery was subsequently used in biotechnology applications for the production of genetically modified glyphosate-resistant plants.

More recently, growing concern for the discharge of residual chemical oxygen demanding (COD) pollutants to receiving waters has resulted in work to establish the biodegradation of other key phosphonates which are currently recalcitrant in waste treatment systems. Typically, this work utilizes laboratory-scale bioreactors seeded with activated sludge and acclimated for several months to feed containing a specific phosphonate as a sole source of carbon or phosphorous. Once microbial activity is achieved, the bioprocess may be characterized and optimized for potential full-scale deployment. In addition, the highly acclimated microorganisms are a novel source of unique biocatalytic reactions.

In this disclosure, we describe the successful development of HMPA-degrading microorganisms and their use in proof-of-concept experiments to show HMPA oxidase activity for the enzymatic oxidation of HMPA to produce biogenic FPA. We also show the production of N-phosphonomethylglycine (glyphosate) from biogenic FPA. The invention provides several pure cultures of HMPA degrading microbes which can be used in bioremediation processes, and biosynthetic processes for the synthesis of FPA and/or glyphosate.

### EXAMPLE 1

### Material & Methods

Chemicals—Hydroxymethylphosphonic acid (HMPA), formylphosphonic acid (FPA) and N-phosphonomethylglycine (glyphosate) were provided by MONSANTO CROP PROTECTION™ and purity exceeded 98%. Glycine was purchased from SIGMA CHEMICAL COMPANY™ (St. Louis, Mo.) and purity exceeded 99%. All microbial culture media were purchased from DIFCO LABORATORIES™ (Detroit, Mich.). Inorganic L-salts medium was prepared as previously described (Leadbetter and Foster, 1958).

Bioreactor A—1.5 liter aerobic stirred batch bioreactor was seeded on May 12, 1998 with fresh mixed liquor collected from the biosystem used to treat glyphosate manufacturing wastes at the Fayetteville, N.C. facility. The mixed liquor was chilled after collection and sent by overnight express shipping to laboratories in St. Louis, Mo. The reactor was maintained at room temperature and pH 7.5±0.5 using NaOH or HCl for adjustment as necessary. Dissolved oxygen levels of 2.0-5.0 mg/L were achieved using an air stone. The bioreactor was fed an inorganic salts medium containing HMPA as a sole source of carbon and energy. The bioreactor was operated from May through December and HMPA-loading was increased step-wise to enrich for HMPA-degrading microorganisms.

Microbial Isolations—Microbial plating studies were conducted with fresh mixed liquor from the bioreactor to isolate potential HMPA degrading microorganisms. Bioreactor samples were diluted in sterile L-salts and spread plated onto L-salts agar, L-salts agar plus 5,000 mg/L HMPA and L-salts agar plus 2,000 mg/L HMPA. Spread plates were incubated for two weeks at 25° C. In addition, microbial isolations were conducted in liquid tube cultures containing L-salts plus or minus HMPA as described above. Microbial growth was compared between L-salts and L-salts plus HMPA. Distinct microbial isolates appearing to grow on HMPA were subcultured on agar plates containing L-salts agar plus 5,000 mg/L HMPA and 5 mg/L yeast extract. Potential HMPA-degrading isolates were also preserved on agar slants plates containing L-salts agar plus 5,000 mg/L HMPA and 5 mg/L yeast extract (YE).

Chemical Analysis—An electrospray HPLC/MS analytical technique was developed for analyses of hydroxy methyl phosphonic acid (HMPA), formyl phosphonic acid (FPA), and glyphosate (N-phosphonomethylglycine). The method used a HEWLETT-PACKARD™ electrospray HPLC/MSD equipped with a HAMILTON™ PRP-X100 (4.1×250 mm) anion exchange column with a flow rate of 1.5 ml/min. water. An acetonitrile (ACN) solvent gradient varied from 0-100% ACN between 3 and 10 minutes to flush any residual cell material. A post-analysis time of 2.5 minutes was used to equilibrate the column to 0% ACN before each injection. All solvents contained 0.1% trifluoroacetic acid (TFA).

The mass spectrometer was operated in the negative ion electrospray mode, scanning between 100 and 650 daltons. All samples were diluted volumetrically 1:5 (or 1:10) by placing 0.1 ml sample and 0.4 (or 0.9) ml deionized water in an autosampler vial. Twenty-five μl injection volumes of standards and samples were used. A piecewise calibration curve was forced through the origin, and four calibration solutions containing 31.25, 62.5, 125, and 250 mg/ml HMPA and FPA were used. Glyphosate concentrations were 250 and 500 mg/ml. The complete calibration curves were checked every 20 samples. were 250 and 500 mg/ml. The complete calibration curves were checked every 20 samples.

The responses of 109, 111, and 168 daltons were used to identify and quantify FPA, HMPA, and glyphosate, respectively. These masses correspond to the (M−H)⁻ formed in the electrospray ionization process of these three molecules. The methodology provided acceptable separation and detection of HMPA and FPA over a concentration range of 0.1-1 mg/ml. Higher concentrations were quantified, if needed, by making further dilutions. This analysis method showed no interference from phosphoric acid or phosphonoformic acid. Overall analysis times were in the range of 12 minutes/sample.

Hydrogenation-Hydrogenation—A 30-50 ml sample of culture fluid from the bioreactor was filtered through a 0.45 micrometer filter to remove bacterial cells. The filtrate was then reacted with 0.5-1.0 g of 10% Pd/Carbon in a pressure vessel. The reaction was incubated overnight at a hydrogen pressure of 80-100 psi at 50-70 degrees C. After the incubation period, the vessel was de-pressurized and the reaction mixture filtered to remove the Pd/carbon. The solution was then analyzed for glyphosate as described above.

### EXAMPLE 2

### Isolation of Microbes with HMPA Degrading Activity

The bioreactor developed HMPA-degrading activity after 10 days of operation and showed complete loss of HMPA after 18 days of operation. In contrast, HMPA concentrations would have reached 1,800 mg/liter after 42 days in the absence of any degradation. The bioreactor maintained levels of HMPA degradation of 96->99% as HMPA loading was increased from 67 up to 300 kg/M3/day for a seven-month period from May through December (Table 1). These results indicate that the mixed cultures would be useful in the biotreatment of wastewater where HMPA was a significant constituent.

TABLE 1
Seven Month Acclimatization of Reactor to HMPA

| | HMPA Loading* | HMPA |
|---|---|---|
| Time Period | (kg/M³/day) | Biodegradation |
| May 22-June 24 | 67 | >99% |
| June 23-July 7 | 100 | >99% |
| July 8-Aug. 30 | 133 | 96->99% |
| Aug. 31-Sept. 10 | 168 | >98% |
| Sept. 11-Oct. 9 | 200 | >98% |
| Oct. 10-Dec. 22 | 300 | >98% |

In response to the observed increase in HMPA-degrading activity in the mixed culture bioreactor, several microbial plating and enrichment studies were conducted to isolate pure cultures of HMPA-degrading microorganisms from the mixed culture reactor. The first three attempts showed no evidence of enhanced microbial growth on media supplemented with HMPA. However, the fourth microbial plating study detected enhanced microbial growth on media containing HMPA after two weeks of incubation at ambient temperature. Eight distinct bacterial isolates, designated 1B-8B, were selected from the agar plates based on colony morphology, color and cellular morphology. These selected cultures were isolation-streaked on L-salts agar containing HMPA and YE to check for purity. It was determined that bacterial isolate 7B was a mix of two strains which were isolated and designated 7B(1) and 7B(2). Bacterial isolates 1B-6B and 8B were pure cultures. Samples of each culture have been deposited according to the terms of the Budapest Treaty as follows:

| Culture | ATCC Deposit Number |
|---|---|
| Mixed sample HMPA-M1 | ATCC PTA-3469 |
| HMPA-1B | ATCC |
| HMPA-2B | ATCC |
| HMPA-3B | ATCC |
| HMPA-4B | ATCC |
| 5B | |
| 6B | |
| 7B(1) | |
| 7B(2) | |
| 8B | |

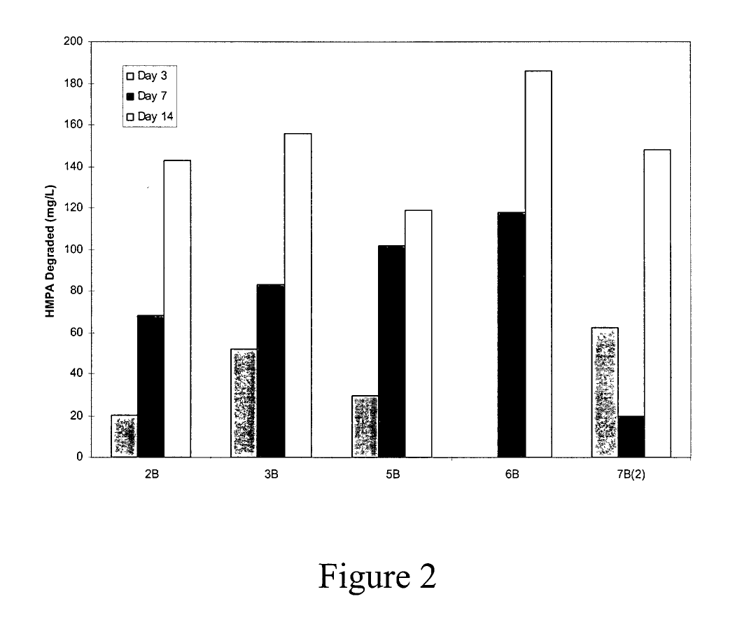

The nine bacterial isolates were incubated in L-salts containing 1000 mg/L HMPA and assayed for HMPA-degrading activity. A significant level of HMPA degradation was detected in several of the bacterial cultures. The degradation of HMPA by bacterial isolates 2B, 3B, 5B, 6B and 7B(2), the five most active cultures, are shown in FIG. 2. Although less than 20% of the HMPA was degraded during the 14-day incubation, this was the first time that HMPA degradation was shown to occur by pure bacterial cultures. Based on these promising results, additional experiments were conducted with these cultures to characterize HMPA and possibly detect biogenic FPA as a degradation intermediate. However, higher rates of HMPA degradation or occurrence of FPA were not detected with these pure cultures.

Since the bioreactor continued to increase in HMPA-degrading activity, an HMPA degradation experiment was conducted with mixed microbial cultures collected from the bioreactor. In this experiment, HMPA concentrations were targeted at 100, 250 and 500 mg/L to examine whether HMPA concentration affected HMPA degradation by the mixed cultures. The results from this experiment (Table 2) show rapid degradation of HMPA at all test concentrations. Actual HMPA exposure concentrations in this experiment were 165, 342 and 595 mg/L. Complete loss of HMPA was detected after 1 day of incubation at the 165 mg/L exposure level and over 98% was degraded after 1 day at the 342 mg/L exposure level. No HMPA was detected in any cultures after 3 and 5 days of incubation.

TABLE 2
Biodegradation of HMPA by a Mixed Microbial
Culture Collected from an Aerobic Stirred Bioreactor

| | | | Actual HMPA mg/l | | |
|---|---|---|---|---|---|
| | | | On a given day | | |
| HMPA | | | of Incubation | | |
| (mg/L) | Microorganisms | Yeast Extract | 1 | 3 | 5 |
| 100 | + | − | 0 | 0 | 0 |
| 100 | + | − | 0 | 0 | 0 |
| 100 | + | + | 0 | 0 | 0 |
| 100 | + | + | 0 | 0 | 0 |
| 100 | − | − | NA | NA | 165.2 |
| 250 | + | − | 5.2 | 0 | 0 |
| 250 | + | − | 4.0 | 0 | 0 |
| 250 | + | + | 4.6 | 0 | 0 |
| 250 | + | + | 4.6 | 0 | 0 |
| 250 | − | − | NA | NA | 342.3 |
| 500 | + | − | 236.8 | 0 | 0 |
| 500 | + | − | 241.5 | 0 | 0 |
| 500 | + | + | 238.5 | 0 | 0 |
| 500 | + | + | 247.5 | 0 | 0 |
| 500 | − | − | NA | NA | 595.4 |
| Water Blank | − | − | NA | NA | 0.0 |

Although the mixed culture bioreactor continued to show increased levels of HMPA degradation throughout this study, this was the first experiment to show high levels of HMPA degradation by mixed microbial cultures diluted 1:1 and assayed outside the bioreactor. These results demonstrated the high level of microbial HMPA-degrading activity achieved by the mixed bacterial cultures collected from the HMPA-acclimated bioreactor.

### EXAMPLE 3

### FPA Production

The high level of HMPA degradation observed by the mixed microbial culture in the previous experiment suggested that significant levels of biogenic FPA, the hypothesized intermediate, may be produced by these highly acclimated cultures. Since it was recognized that FPA levels were likely to be low due to rapid further degradation, glycine was utilized as an aldehyde-trap to slow further degradation of FPA and increase the likelihood of detection. The experiment was conducted with the HMPA-degrading mixed microbial culture samples at an HMPA exposure concentration of 360 mg/L and glycine was added to the media (1:1 molar ratio to HMPA) as an aldehyde trap to bind any biogenic FPA. In addition, one sample was incubated with HMPA and glycine was hydrogenated to potentially convert any biogenic FPA directly to N-phosphonomethylglycine.

The results from this experiment are the first report of the occurrence of FPA as a microbial degradation product of HMPA (Table 3). Biogenic FPA was detected in samples one and two at concentrations ranging from 17 to 65 mg/L after 6 and 24 hours of incubation. FPA was also detected at similar concentrations in sample three which lacked glycine as an aldehyde trap.

In all three samples, the highest levels of FPA were detected after 6 hours of incubation and levels dropped later in the experiment. This suggested that FPA occurred as a transient intermediate during high rates of HMPA biodegradation and FPA underwent rapid bacterial degradation when HMPA levels were depleted. No HMPA or FPA were detected in a control sample not exposed to HMPA. These results clearly showed that FPA was produced as a bacterial oxidation product of HMPA and proved the concept that FPA can be produced enzymatically from HMPA.

TABLE 3
Bacterial Degradation of HMPA and Occurrence of FPA and Glyphosate*

| Mixed | | | | | | | | |
|---|---|---|---|---|---|---|---|---|
| Culture | Incubation | | | | | HMPA | FPA | Glyphosate |
| Sample | (hr) | HMPA | Bacteria | Glycine | Hydrogenated* | mg/L | Mg/L | mg/L |
| 1 | 0 | + | + | + | − | 363 | 0 | − |
| | 6 | + | + | + | − | 332 | 57 | − |
| | 24 | + | + | + | − | 245 | 20 | − |
| | 72 | + | + | + | + | 94 | 0 | 81 |
| 2 | 0 | + | + | + | − | 344 | 0 | − |
| | 6 | + | + | + | − | 311 | 65 | − |
| | 24 | + | + | + | − | 258 | 17 | − |
| | 72 | + | + | + | − | 59 | 18 | − |
| 3 | 0 | + | + | − | − | 358 | 0 | − |
| | 6 | + | + | − | − | 299 | 65 | − |
| | 24 | + | + | − | − | 253 | 15 | − |
| | 72 | + | + | − | − | 0 | 0 | − |
| 4 | 0 | − | + | + | − | 0 | 0 | − |
| | 6 | − | + | + | − | 0 | 0 | − |
| | 24 | − | + | + | − | 0 | 0 | − |
| | 72 | − | + | + | − | 0 | 0 | − |

*Sample was hydrogenated overnight prior to LC/MS analysis

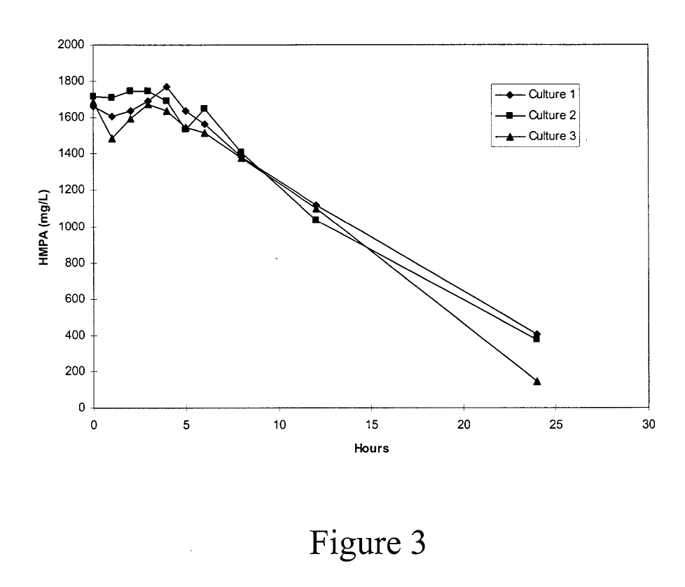

Experiments were conducted using higher biosolids concentrations, higher HMPA concentrations and more frequent sampling to characterize HMPA degradation and confirm the occurrence of biogenic FPA. The biodegradation of HMPA by five-fold concentrated microbial mixed culture samples exposed to 1,700 mg/L HMPA are shown in FIG. 3. All three samples showed a six hour lag phase followed by rapid and linear degradation of HMPA between 8 and 24 hours of incubation.

The occurrence of biogenic FPA from microbial oxidation of HMPA in this same experiment is shown in Table 4. Biogenic FPA was detected in all three mixed culture samples at concentrations ranging from 35-50 mg/L during 2-4 hours of incubation. Although FPA was not detected in samples one and three after 24 hours, it was detected throughout the experiment in sample two.

TABLE 4
Occurrence of Biogenic FPA in Mixed Microbial
Cultures Exposed to HMPA

| | FPA mg/l | | |
|---|---|---|---|
| | Mixed Culture | Mixed Culture | Mixed Culture |
| Hours | Sample 1 | Sample 2 | Sample 3 |
| 0 | 0 | 0 | 0 |
| 1 | 0 | 0 | 0 |
| 2 | 48.5 | 38.5 | 36.5 |
| 3 | 38.8 | 42.7 | 54.3 |
| 4 | 38.3 | 35.2 | 40.4 |
| 5 | 0 | 0 | 0 |
| 6 | 21.3 | 31.7 | 0 |
| 8 | 0 | 31.7 | 0 |
| 12 | 0 | 21.6 | 12.8 |
| 24 | 0 | 19.8 | 0 |

The aerobic stirred bioreactor was operated at progressively higher concentrations of HMPA, reaching a maximum loading of 300 kg/M3/day of HMPA after seven months of acclimation. The bioreactor maintained 96-99% removal of HMPA throughout this study, as described earlier (see Table 1). It is noteworthy that FPA was detected intermittently in the bioreactor at concentrations ranging from 4-18 mg/L only in the final three weeks of this investigation. The occurrence of FPA in the bioreactor demonstrated how highly enriched the mixed culture had become after seven months of exposure to HMPA as sole source of carbon and energy.

### EXAMPLE 4

### Glyphosate Production from FPA Activity

Proof-of-concept for the production of N-phosphonomethylglycine (glyphosate) from biogenic FPA was achieved by incubating an HMPA-degrading culture with HMPA and glycine (1:1 molar ratio) and hydrogenating the culture filtrate directly. Hydrogenation of the filtrate from mixed culture sample 3 after 72 h of incubation resulted in the production of 81 mg/L of N-phosphonomethylglycine (glyphosate) from biogenic FPA (Table 3).

### EXAMPLE 5

### Biodegradation of FPA

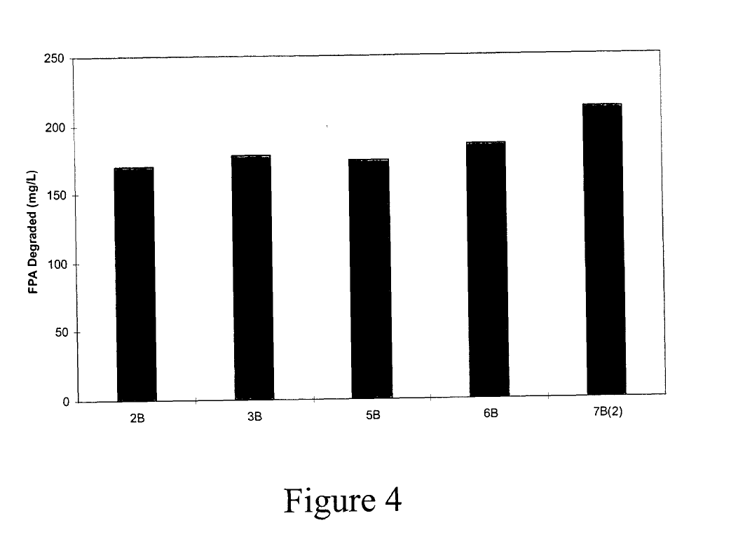

Since FPA in the previous experiments appeared transient and did not accumulate to high concentrations, it was assumed that FPA was readily degradable by the bacterial cultures. An experiment was conducted with authentic FPA to determine whether HMPA-degrading bacterial isolates 2B, 3B, 5B, 6B and 7B(2) degraded FPA (FIG. 4). The five bacterial isolates exposed to 212 mg/L of authentic FPA degraded 80-100% of the FPA within 24 hours. Comparison to FIG. 2 shows that FPA was degraded much faster than HMPA by these bacterial isolates. These results confirmed that FPA undergoes rapid bacterial degradation and explain why FPA did not accumulate to high concentrations in the previous experiments.

In summary, these experiments were the first to show that formylphosphonic acid (FPA) is produced enzymatically by bacterial oxidation of hydroxymethylphosphonic acid (HMPA). Furthermore, proof-of-concept was shown for the production of N-phosphonomethylglycine (glyphosate) from biogenic FPA.

### EXAMPLE 6

### Future Experiments

The above proof-of-concept studies have laid the groundwork for additional work to advance the development of HMPA oxidase for production of biogenic FPA, a key intermediate in new chemistry to manufacture glyphosate. Planned experiments include:

1) Characterize HMPA Oxidase—Pulse-chase studies with 14C-HMPA will be used to further characterize the rates of biogenic FPA production. This information would be useful in beginning to characterize the enzyme. Additionally, HMPA derivatives will be tested in order to elaborate the substrate specificity of the enzyme. When some information about the enzyme has been obtained, the HMPA oxidase will be purified and further characterized. Purification will lead to cloning and sequencing the enzyme as a first step towards the development of an engineered HMPA biocatalyst.

2) Novel Mutants—HMPA-degrading bacterial cultures will be mutagenized and screened for mutants which degrade HMPA (HMPA⁺), but accumulate FPA (FPA⁻). These mutants would provide increased accumulation of FPA, thus obviating the need for continuous FPA collection or trapping strategies.

3) Bacterial Characterization—The bacterial isolates listed above have been forwarded to a commercial lab for identification tests. A variety of biochemical tests will be performed in order to identify the species and the genus of organism. The tests may include: assessment of growth conditions, culture appearance, cell appearance and staining characteristics, optimal temperature and pH growth range, oxygen requirements, antibiotic sensitivity testing, tests for catalase and oxidase, amino acid utilization as carbon source, nitrogen fixation, motility test, and a variety of commercially available tests, for example those from MICRO-ID™, OXYFERM™, ENTEROTUBE™ and BIOLOG GN MICROPLATE™. Total protein profiles will be compared by SDS-PAGE.

Fatty acid analysis can be performed, for example, by Microbial ID (MIDI) of Newark, Del. who uses the high resolution MIDI Sherlock System™ to identify fatty acids with high resolution gas chromatography. The same laboratory can also sequence the 16S gene and compared it against the proprietary MICROSEQ™ database (PE APPLIED BIOSYSTEM™). Other possible commercial laboratories and identification systems include QUALICON™, Inc. (Wilmington Del.) which offers the RIBOPRINTER™ MICROBIAL IDENTIFICATION SYSTEM and the University of Guelph which uses four automated microbial identification systems (MICROSCAN WALKAWAY 40™, BIOLOG™, REPLIANALYZER™ and MICROBIAL IDENTIFICATION SYSTEM™).

With these tests, it is hoped that the genus and species of microorganism with HMPA oxidase activity will be identified, or at least additional identifying characteristics for the isolates will be provided.

All patents and articles cited herein are expressly incorporated by reference and are re-listed here for convenience.

Balthazor, T. M. and L. E. Hallas. 1986. Glyphosate-degrading microorganisms from industrial activated sludge. Appl. Environ. Microbiol. 51:432-434.

Carson, D. B., M. A. Heitkamp and L. E. Hallas. 1997. Biodegradation of N-phosphonomethyliminodiacetic acid by microorganisms from industrial activated sludge. Can. J. Microbiol. 43:97-101.

Hallas, L. E., E. M. Hahn and C. Korndorfer. 1988. Characterization of microbial traits associated with glyphosate biodegradation in industrial activated sludge. J. Ind. Microbiol. 3:377-385.

Hallas, L. E., W. J. Adams and M. A. Heitkamp. 1992. Glyphosate degradation by immobilized bacteria: Field studies showing removal of glyphosate from industrial wastewater. Appl Environ. Microbiol. 58:1215-1219.

Heitkamp, M. A., W. J. Adams and L. E. Hallas. 1992. Glyphosate degradation by immobilized bacteria: Laboratory studies showing feasibility for glyphosate removal from wastewater. Can J. Microbiol. 38:921-928.

Hallas, L. E. and M. A. Heitkamp. 1995. Microbiological treatment of chemical process wastewater. Chapter 10 in “Microbiological Transformation and Degradation of Toxic Organic Chemicals” (Editors: C. E. Cerniglia and L. Y. Young), pp. 349-387, John Wiley

Leadbetter, E. R. and J. W. Foster. 1958. Studies on more methane-utilizing bacteria. Arch. Mikrobiol. 30:91-118.

Murthy, D. V. S., R. L. Irvine and L. E. Hallas. 1988. Principles of organism selection for the degradation of glyphosate in a sequencing batch reactor, p. 267-274. In J. M. Bell (ed.), 43rd Annual Purdue Industrial Waste Conference. Lewis Publishers, Chelsea, Mich.

ES5044479; GB1076244; U.S. Pat. Nos. 3,799,580; 3,835,000; 3,853,530; 3,927,080; 3,969,398; 3,977,860; 3,988,142; 4,065,491; 4,140,513; 4,315,765; 4,369,142; 4,405,531; 4,422,982; 4,481,026; 4,486,358; 4,486,359; 4,507,250; 4,568,432; 4,810,426; 4,840,659; 4,851,159; 4,983,764; 5,180,846; 5,266,722; 5,292,936; 5,578,190; 5,602,276; 5,679,843; 5,693,593; 5,874,612; 5,912,209; 5,935,905; 5,948,937; 5,985,794; 5,994,269; 5,998,332; 6,005,140; 6,054,608; 6,083,878;

What is claimed is:

1. A method of bioremediating wastewater or soil contaminated with a HMPA derivative, comprising exposing wastewater or soil to one or more HMPA degrading microbes under conditions that allow the degradation of HMPA derivative.

2. The method of claim 1, wherein said one or more HMPA degrading microbes are selected from the group consisting of ATCC ______, ATCC ______, ATCC ______, ATCC ______ and ATCC PTA-3469.

3. A method of producing an amino phosphonate derivative, comprising condensing a biogenic FPA derivative and a nitrogen-containing compound to form a condensation product, and subsequent conversion of the condensation product to form an amino phosphonate derivative.

4. The method of claim 3, wherein the amino phosphonate derivative is a glyphosate derivative.

5. The method of claim 3, wherein the biogenic FPA derivative is produced by one or more microbes selected from the group consisting of ATCC ______, ATCC ______, ATCC ______, ATCC ______ and ATCC PTA-3469.

6. The method of claim 4, wherein the conversion is by hydrogenation.

7. The method of claim 4, wherein the conversion is by hydrogenation, oxidation, carboxymethylation or combinations thereof.

8. The method of claim 3, wherein the amino phosphonate derivative is hydroxyethylaminomethylphosphonic acid or salt or ester thereof.

9. The method of claim 8, wherein the conversion of the condensation product to form hydroxyethylaminomethylphosphonic acid or salt or ester thereof is by hydrogenation.

10. The method of claim 8, wherein the hydroxyethylaminomethylphosphonic acid or salt or ester thereof is converted to a glyphosate or salt or ester thereof by oxidation.

11. A method of producing glyphosate, comprising condensing a biogenic FPA derivative and a glycine to form a condensation product, and subsequent conversion of the condensation product to form a glyphosate.

12. A method of producing glyphosate, consisting essentially of condensing a biogenic FPA derivative and a glycine to form a condensation product, and subsequent conversion of the condensation product to form a glyphosate.

13. The method of claim 11 or 12, wherein the conversion of the condensation product to form a glyphosate is by hydrogenation.

14. The method of claim 11 or 12, wherein the conversion is by hydrogenation, oxidation, carboxymethylation or combinations thereof.

15. A method of producing glyphosate by condensing a biogenic FPA derivative and alkanolamine and subsequent conversion of the condensation product to form glyphosate.

16. The method of claim 15, wherein the alkanolamine is ethanolamine.

17. A method of producing glyphosate by condensing a biogenic FPA derivative and ethanolamine and subsequent conversion of the condensation product to form glyphosate.

18. A method of producing a biogenic FPA derivative, the method comprising incubating an HMPA derivative with one or more HMPA degrading microbes under conditions that allow the degradation of said HMPA derivative to form an FPA derivative and collecting the FPA derivative.

19. The method of claim 18, wherein said one or more HMPA degrading microbes are selected from the group consisting of ATCC ______, ATCC ______, ATCC ______, ATCC ______ and ATCC PTA-3469.

20. A method of isolating a pure culture of HMPA degrading bacteria, comprising the following sequential steps:
a) seeding a reactor with a mixed microbial population;
b) maintaining the reactor with HMPA as the sole carbon source in order to select HMPA degrading bacteria; and
c) purifying the HMPA degrading bacteria.
